(19) United States
(12) Patent Application Publication
(10) Pub. No.: US 2026/0323170 A1
(43) Pub. Date: Oct. 15, 2026

(54) IMAGING DEVICE FOR CONTROLLED ENVIRONMENTAL AGRICULTURE SPACES

(71) Applicant: CANON VIRGINIA, INC., Newport News, VA (US)

(72) Inventors: Sean M. DeHart, Hampton, VA (US); Paul Risque, Williamsburg, VA (US); Connor Leschinsky, Newport News, VA (US); Sean Ison, Hampton, VA (US); Jeffrey T. Sharps, Yorktown, VA (US); Jacob Landes Sheets, Lanexa, VA (US); Thomas Ortmann, Williamsburg, VA (US); Joseph Milhorn, Newport News, VA (US)

(21) Appl. No.: 19/702,267

(22) Filed: Jun. 9, 2026

Related U.S. Application Data
(63) Continuation of application No. PCT/US2024/059323, filed on Dec. 10, 2024.
(60) Provisional application No. 63/608,689, filed on Dec. 11, 2023.

(51) Int. Cl. H04N 23/90; G01B 11/06; G01B 11/22; G03B 7/26; G03B 17/56; H04N 23/69; H04N 23/695

(52) U.S. Cl. CPC H04N 23/90; G01B 11/0608; G01B 11/22; G03B 7/26; G03B 17/561; H04N 23/69; H04N 23/695

(57) ABSTRACT

The present disclosure relates to agriculture monitoring in a gantry system using one or more stationary of mobile cameras, including in an array.

### CROSS REFERENCE TO RELATED APPLICATIONS

This application is a Continuation of International Patent Application No. PCT/US 2024/059323, filed Dec. 10, 2024, which claims the benefit of priority from US Provisional Patent Application Ser. No. 63/608,689 filed on Dec. 11, 2023, where is incorporated herein by reference in its entirety.

### BACKGROUND

### Field of the Disclosure

The present disclosure relates to the field of agriculture monitoring.

### Description of the Related Art

Controlled indoor agriculture spaces are largely monitored by human workers. This is inefficient and prone to human error. Monitoring these spaces with imaging hardware reduces the manpower load and also allows for computer analysis which can be more rigorous and consistent in monitoring the space.

### SUMMARY

According to the present disclosure, an image capturing system is provided and includes a carriage and at least one camera carried by the carriage. At least one motoring wheel to drive the carriage along a rail or a tensioned cable, wherein the at least one camera is configured to capture images with different angle or field of view.

According to another embodiment, the the at least one camera captures a plant or a vegetation, and at least one camera is configured to capture the images with different angle or field of view based on a growth of the plant or the vegetation.

According to a further embodiment, the at least one camera captures a plant or a vegetation and the carriage carries a first camera and a second camera having different field of view, and whether the first camera performs a capturing of the plant or the vegetation or the second camera performs a capturing of the plant or the vegetation is determined based on a growth of the plant or the vegetation.

According to another embodiment, the at least one camera captures a plant or a vegetation, wherein the at least one camera can be tilted and a range that the at least one camera is tilted during a capturing of the plant or the vegetation is determined based on a growth of the plant or the vegetation.

In another embodiment, at least one camera captures a plant or a vegetation and an angle of the carriage is changeable, and a range that the angle of the carriage is changed during a capturing of the plant or the vegetation is determined based on a growth of the plant or the vegetation.

In yet another embodiment, the image capturing system includes a detector to detect height of at least one plant and the detector including depth sensor. The image capturing system further comprises a collision avoidance sensor and a wireless charger configured to charge a battery in the carriage. In certain embodiment, the wireless charger is located at an end of the rail.

In another embodiment, an image capturing device is provided an includes a carriage and at least one camera carried by the carriage and at least one motoring wheel to drive the carriage along a fixed path, wherein the at least one camera is configured to capture images with different angle or field of view.

In certain embodiments, the fixed path is a rail or a tensioned cable and the one or more cameras are movable in a direction orthogonal to direction the carriage travels on the fixed path.

In another embodiment, the one or more cameras are an array of cameras and the array of cameras can be fixed in position or tiltable.

These and other objects, features, and advantages of the present disclosure will become apparent upon reading the following detailed description of exemplary embodiments of the present disclosure, when taken in conjunction with the appended drawings, and provided claims.

### BRIEF DESCRIPTION OF THE DRAWINGS

The accompanying drawings, which are incorporated herein and form part of the specification, illustrate various embodiments, objects, features, and advantages of the present disclosure.

FIGS. 1A and 1B are conceptual drawings of a imaging device and system configured to move along a rail that is attached to a support structure.

FIGS. 2A and 2B are conceptual drawings of a stationary imaging device attached to a support structure in a manner to allow complete coverage of the vegetation below.

FIGS. 3A and 3B are conceptual drawing of a stationary camera array wherein the cameras are tiltable.

FIG. 4 is a drawing of a moveable, bendable arm that supports one or more cameras.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative exemplary embodiments. It is intended that changes and modifications can be made to the described exemplary embodiments without departing from the true scope and spirit of the subject disclosure as defined by the appended claims.

### DETAILED DESCRIPTION OF EXEMPLARY EMBODIMENT

The present disclosure has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

It is noted that the embodiments described herein can be used in combination with any of the embodiments provided in U.S. patent application Ser. No. 17/272,079, filed Feb. 26, 2021 as the national phase entry of Patent Cooperation Treaty Application No. PCT/US 19/48602 filed Aug. 28, 2019 and claiming priority to U.S. Provisional Patent Application Nos. 62/818,432 filed Mar. 14, 2019 and 62/725,137 filed Aug. 30, 2018, each of which is hereby incorporated herein in their entirety.

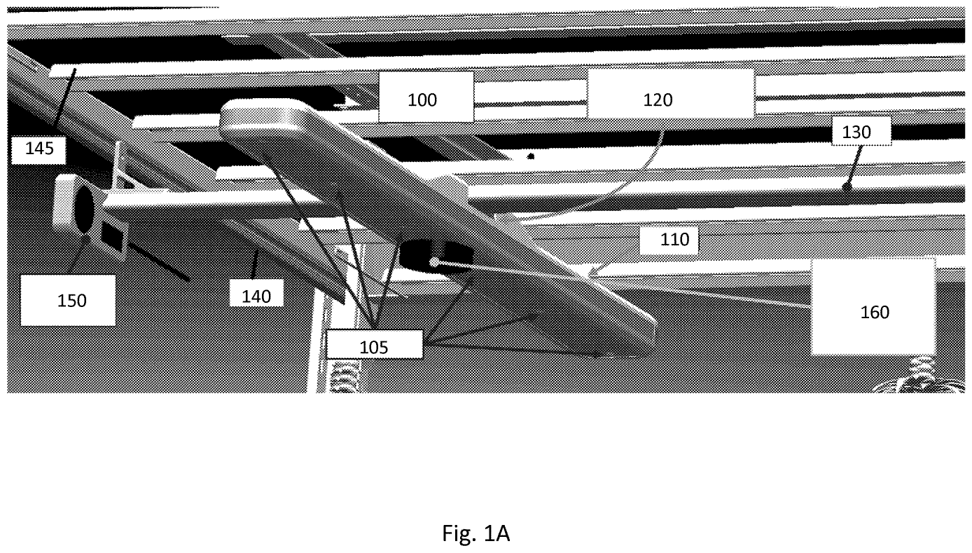

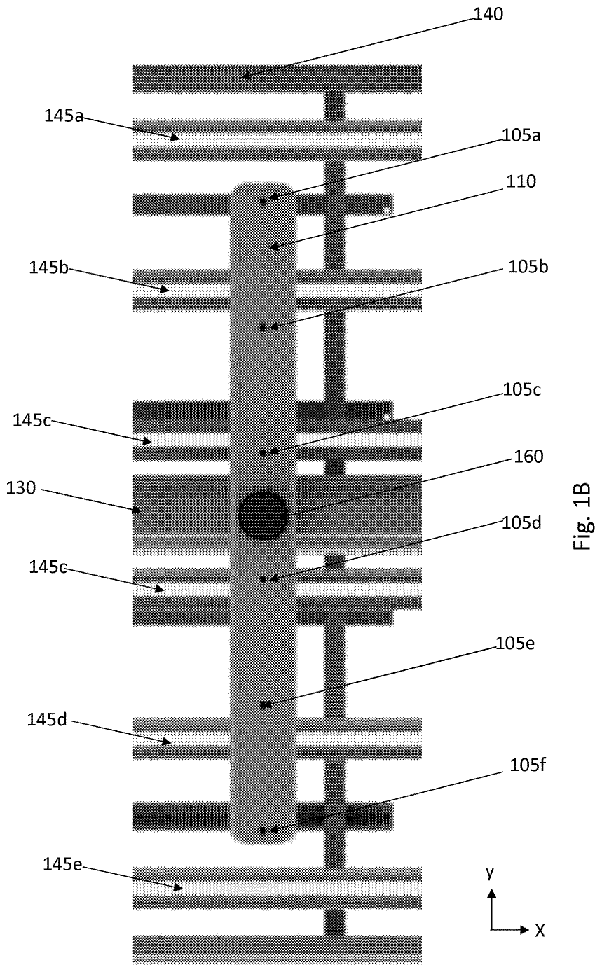

FIG. 1A is a conceptual drawing of an imaging device and system configured to capture one or more images (video and/or still images) of vegetation growing in a controlled environment agricultural space. As used herein the term vegetation includes one or more plants or crops that are being grown in one or more grow containers. In one embodiment the controlled environment agricultural space is a green house or a grow house where vegetation are grown in a manner whereby all environmental variables associated with growing plants/crops are controlled by individuals. In certain instances, the control of the environment is performed manually by a user who physically checks and modifies environmental variables such as light, temperature, humidity, pH, water applications and the like. In other embodiments, these environmental variables are selectively controllable by one or more computer applications that execute on one or more computing devices (e.g. local server, cloud server, etc.) which automate certain aspects of controlling these environmental variables to meet predefined conditions and/or standards.

The system described herein include one or more embodiments of imaging hardware system that captures images for agricultural monitoring of plants being grown in indoor grow location that have controllable environments. Each hardware configuration encompasses at least one RGB camera. For purposes of the present disclosure, embodiments of the imaging system is mountable on a support structure such as a gantry system, which provides single or multi-level racks for indoor agriculture operations. While the disclosure will refer to one or more gantries as the type of support structure, it should be understood that any type of support structure can be used so long as the imaging system has at least a partial overhead view of the vegetation being grown in the indoor grow location.

The embodiment illustrated in FIG. 1A represents an imaging system includes an imaging device 100 formed from a housing 110 (e.g. a carriage) having a movement mechanism 120 (at least one motor wheel) that is connected to and is configured to move along a track 130 that is attached to a gantry 140. The housing 110 is formed from a material that seals and insulates internal electronic component from the grow environment to avoid water from entering the housing and to ensure that changes in temperature do not negatively impact the components of the imaging device.

The exemplary gantry 140 includes one or more light emitting elements 145 that are positioned over the vegetation being grown in the one or more grow containers (not shown) supported by a shelf or other elements of gantry 140. The system 100 includes an imager dock 150 that couples with or otherwise connects to the housing 110 of the imaging device 100. The imager dock 150 is supplied with local power and is configured to provide the supplied power to the imaging device 100 to power the various electrical components contained therein. The provision of power may be provided in any known manner of power transfer including direct power transfer or wireless power transfer. In one embodiment, the imager dock 150 includes a wireless charger can be used to provide power to the imaging device. In some embodiments, the imager dock 150 is located at an end of the track 130 (e.g. rail or cable) so that the imaging device 100 including the one or more cameras does not block the light being emitted from light sources 145 from reaching the vegetation while the imaging device is connected (or otherwise coupled to) the imager dock 150 during wireless charging operation. The position of the imager dock 150 is not limited to the end of the rail, but instead can be determined based on the specific configuration of the vegetation, light sources and the imaging device travel path, such that any position where the imaging device does not block the light can be adopted. In other embodiments, the charging functionality implemented in and provided by the imager dock 150, may be included as part of the track 130 (e.g. the rail) along which the imaging device 100 travels. In this embodiment, the movement mechanism 120 would include electrical contacts or wireless charging circuits to match with those provided in the track 130.

In another embodiment, the imager dock 150 includes data transfer functionality that, upon the imaging device 100 docking therewith, initiates data acquisition of data captured by the imaging device 100. The imager dock 150 further includes communication circuitry and transfers the acquired image data to an external computing system (e.g. server and/or cloud server) where the acquired image data can undergo image analysis to identify one or more characteristics about the vegetation captured by the imaging device 100. In certain instances, the image analysis performed on the images of the vegetation captured by the imaging device 100 includes determining health of the vegetation by using the captured image to determine whether individual plants are healthy or diseased or distressed. This also includes determining whether the plant health is impacted by pests such as mites. These are merely examples of image analysis operations that may be performed on the image data of the vegetation captured by the imaging device 100 and should not be considered limiting as any type of image analysis may be performed by a server or cloud server that receives image data captured by the imaging device 100 and transmitted thereto by the imager dock 150.

FIG. 1A illustrates the movement mechanism 120 of the imaging device as being a linear motion system. In this embodiment, the mechanism 120 includes a motorized driving wheel that contacts the track 130 (as shown herein the track is rail) and, in response to a movement control signal, causes the imaging device 100 to move along the track 130. Similarly to the housing 110, the movement mechanism is formed such that an outer shell protects any wheels or drive mechanism contained therein and which is used to move the imaging device along a length of track 130 as described herein. The imaging device 100 also includes a collision sensor 160 that can detect proximity to objects to avoid collisions between the imaging device 100 and other objects. In one embodiment, the collision sensor 160 is LIDAR sensor. The collision sensor 160 provides positional awareness along the rail and use LiDAR for collision avoidance by observing the walls of the room and/or aspects of the support structure 140. The inclusion of the collision sensor 160 advantageously can provide signals corresponding to detected objects which can be used control movement of the imaging device 100 along the track 130 to stop its motion to avoid hitting or damaging plants if they got too tall or walls of the grow environment or persons charged with working in and around the vegetation being grown. The collision sensor 160 can detect distance between the imagining device 100 and any of the vegetation that is growing and generate control signals that modify the movement of the imaging device 100 or to provide notification information to persons responsible for working on the vegetation being grown. As such, the collision sensor 160 advantageously provides a safety function for the people performing maintenance on the grow system to prevent the imaging device 100 from bumping into them, or pinching fingers, etc. By using the signals sensed by the collision sensor 160 to avoid obstacles advantageously reduces the potential damage to the imaging device 100 in the grow environment. Other advantages provided by the avoidance sensing is reduce the risk of spreading disease or contamination between various plants and the imaging device 100. As used herein, obstacles include but are not limited to vegetation, support structures/walls, human workers and/or any other component of the grow environment (e.g. lights, fans, ductwork, etc.). In another embodiment, where the track 130 provides a path for the imaging device 100 to traverse to other regions of the grow environment and that path overlaps with other imaging devices, the collision sensor 160 may use LIDAR to prevent collisions between imaging devices by using the scans from LIDAR to identify the position if the internal encoder from the motor of the movement mechanism 120 loses sync. The motor in the movement mechanism 120 includes an encoder to verify that it can move the imaging device 100 as intended and the LIDAR acts as an external feedback to the motor in the imaging device 100 thereby allowing the system to be closed loop in positioning.

The imaging device 100 includes one or more cameras generically referenced by reference numeral 105. In certain embodiments, the camera(s) 105 include an array of cameras. In one embodiment, camera(s) 105 are stereoscopic cameras. The imaging device 100 includes an array of at least one camera 105 which is caused to travel along a dedicated path (e.g. track 130). In some embodiments, the path can be a rail. In other embodiments the path may be a rigid structure such as a beam, or a flexible structure such as a tensioned cable. The imaging device 100 with its camera array 105 can travel along the path set out by either a rigid or flexible structure. Each camera array 105 includes at least two cameras and each of the at least two cameras are configured to capture different fields of view. In one embodiment a first camera of each camera array captures a first field of view and a second camera in the array captures a second field of view that is narrower than the first field of view. The camera array on a respective imaging device 100 may be in an M-N configured where M represents a number of cameras and associated fields of view capturable thereby and N represents a number of camera locations on the image device 100. For example, the array includes any number of cameras from 2-8 or 2-16 indicating that at each camera location, there are two cameras that can capture the two different size fields of view where one is narrower than the other. It should be noted that the number of cameras is limited only by the structure and size of the imaging device 110.

In certain embodiments, the imaging system in FIG. 1A is a rail-based camera system where the system moves along the space and the camera moves at a 90°angle to the system motion to cover more space with a single or smaller number of cameras. For instance, using FIG. 1 as an example, in certain embodiments, the imaging device can move along a rail or tensioned cable in an X direction, while one or more cameras present in the imaging device can move in a Y direction so that the one or more cameras cover a wider area than would be observed if the one or more cameras remained stationary within the imaging device. Instead of having multiple static cameras in fixed position within the imaging device 100, one or more cameras can move linearly inside the imaging device which advantageously enables inclusion of a single higher quality/cost camera that can provide the same functionality as the plurality of individual cameras described herein. While this single/multiple cameras can move inside the housing 110 of imaging device 100. It also can tilt at an angle if necessary to extend the FOV (field of view) coverage. The tilting motion of the camera(s) can be done through the use of a motor, solenoid, mechanical cam(s), follower(s), and roller(s).

FIG. 1B is a bottom view of the housing 110 of the imaging device 100 shown in FIG. 1A that is mounted on the support structure 140 that includes a plurality of light sources 145a-145e that run parallel to the track 130 to which the imaging device 100 is connected. As shown herein, the imaging device housing 110 positioned perpendicular to each of the light sources 145a-145e and the track 130 which are parallel with respect to one another. In exemplary operation, the imaging device 100 moves in the x-direction. However, this is shown for purpose of example only. It should be noted that the track 130 could be perpendicular to the light sources 145a-145e allowing the imaging device to move in the y-direction. Alternatively, the light sources 145a-145e may extend perpendicular to the track 130 and thus be parallel to the imaging device 100.

FIG. 1B further illustrates the orientation and positions of the various cameras 105a-105f contained within the housing 110 of the imaging device. As shown herein, there are six camera positions extending in along the y-axis of the housing 110 of the imaging device. In certain embodiments, each camera position is an aperture through which at least two cameras (or camera lens) extend therethrough enabling capture of the vegetation present in the defined field of view of the respective camera. In another embodiment, the camera positions represent a location on the housing 110 that an external camera can be positioned or otherwise secured thereto.

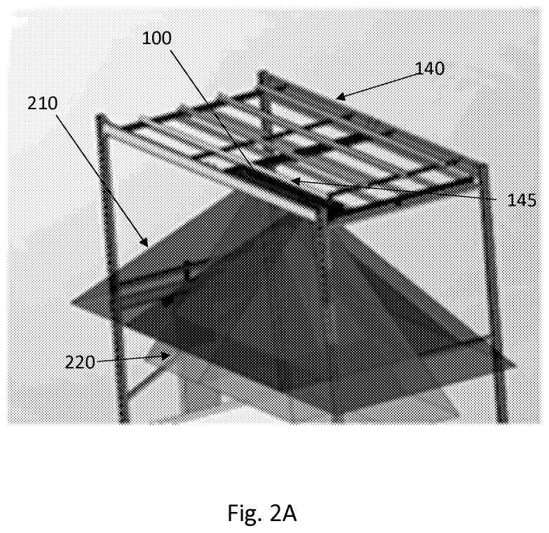

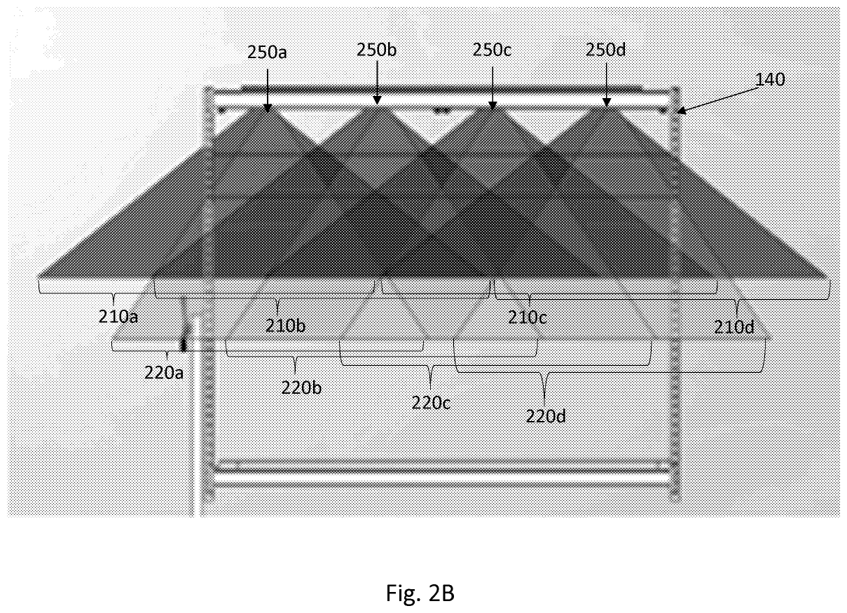

FIGS. 2A and 2B illustrate an alternate embodiment of an imaging device 100 as described in FIGS. 1A and 1B. As shown herein, the imaging device 100 is static and the array of cameras on the imaging device are positioned behind the light sources 145 so as not to restrict light being emitted from the light sources 145 from reaching the vegetation growing below the imaging device 100. In one embodiment, an array of HD narrow angle cameras can be used to cover the full grow space when the plants are young and far away from the cameras. FIG. 2A is top down isometric view of the support structure 140 supporting a single imaging device 100 positioned between a light source 145 and an edge of the support structure 140. This is shown for exemplary purposes and a number of imaging devices 100 may be incorporated into this embodiment by being position between parallel light sources 145 and/or edges of the support structure 140. For example, in the support structure 140 illustrated herein, up to five static imaging devices 100 may be incorporated. In an alternative embodiment, the support structure 140 can include at a first type of imaging device 100 (e.g. a static imaging device) and a second, different type of imaging device (e.g. a moveable imaging device as described in FIGS. 1A & 1B).

As described above the imaging device includes a camera that captures images within a first field of view. In one embodiment, the camera is wide angle camera that has a field of view set to capture the entire grow space closer to the cameras when the plants grow taller. In another embodiment the imaging device includes one or more cameras having different fields of view. This is illustrated in FIG. 2A whereby the imaging device provides an exemplary embodiment depicting stationary cameras with more than one field of view. In some embodiments, an array comprising both HD narrow angle and wide angle cameras can be included at various positions on the imagining device similarly to the arrangement shown in FIG. 1B and are installed within an agricultural area to image the plants below. In such an instance, it is important that the camera array be positioned in a manner such that grow lights are not impeded in providing light to the plants. In another embodiment, the cameras of the array may either vary their fields of view or multiple cameras with differing fields of view may be used to ensure sufficient imaging coverage is maintained while the plants grow. As shown in FIG. 2A, the imaging device 100 is configured to capture a first field of view 210 and a second field of view 220 wherein the second field of view 220 is narrower than the first field of view 210. In one embodiment a first field of view 210 ranges between 100 and 150 degrees and the second field of view 220 ranges between 50 and 100 degrees. In other embodiments, the first field of view 210 is 120 degrees and the second field of view 220 is 75 degrees.

FIG. 2B is a side view of the support structure shown in FIG. 2A. From this viewpoint, a plurality of camera positions 250a-250d are illustrated and shown as including in the imaging device. Each of the cameras at positions 250a-250d capture first fields of view 210a-210d and second fields of view 220a-220d, the second fields of view being narrower than the first fields of view. Because of the arrangement of the camera positions on the imaging device 100, and the different fields of view able to be captured by each respective camera, there are overlapped regions of the grow area that are imaged.

Capturing images from different fields of view provides certain advantages. For example, depending on the position and orientation of X leaf (tilted towards or tilted away) compared to the camera view, as well as the position of other leaves either in front of or behind the X leaf, one image from perspective Y may not capture the complete leaf. Or if the camera does capture the entire X leaf, if the leaf is angled away from the camera view, then the resolution of defects can be affected. Capturing images of plants from multiple perspectives helps mitigate the variability in leaf orientation, as well as give additional chances to see deeper into the plant canopy. For example, as a first imaging device might be blocked by a full view of leaf X, but a second different imaging device might be able to see leaves behind leaf X due to viewing leaf X edge wise, and it therefore not filling the FOV. Other advantages from capturing overlapped coverage areas of a grow tray allows better depth calculations to find the distance between the camera and plant height. This arrangement and ability to capture from different field of views advantageously allows for expansion of stereoscopic imaging to make three dimensional images of the vegetation being captured. Furthermore, using multiple stereoscopic images reduces triangulation errors by providing complementary measurements of the same target object (e.g. plant).

A static array of cameras as shown in FIGS. 2A and 2B offers a very low complexity system to cover an entire grow space. While a large number of cameras may be necessary to cover the space, the static locations make the imaging results very consistent. Number and field of view of the cameras could be optimized for different spaces. Because there is no motion component, cable management is not an issue, so there is no need for wireless communication.

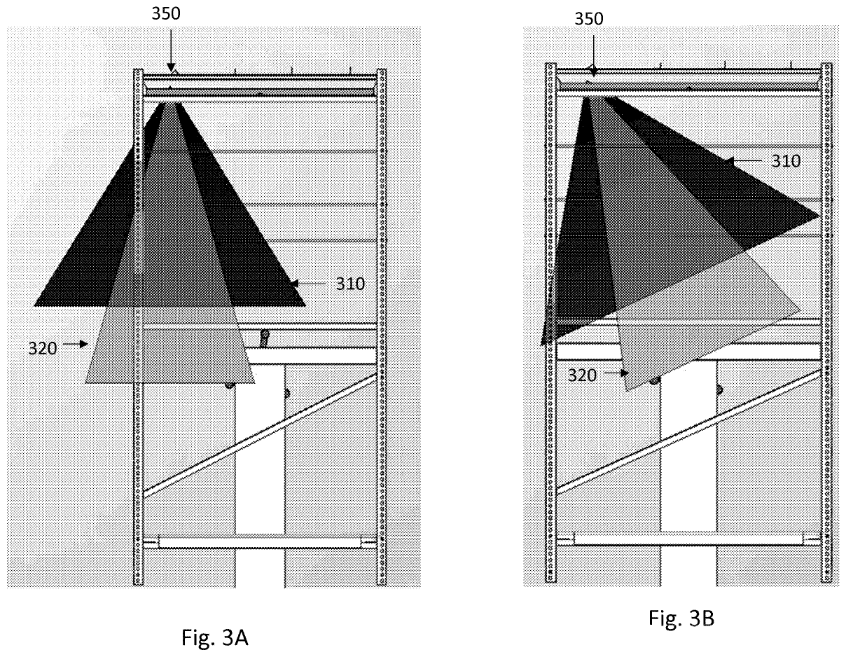

FIGS. 3A and 3B illustrate an alternate embodiment of the static camera arrays illustrated in FIGS. 2A and 2B. As shown herein, the imaging device 100 includes at least one set of tiltable cameras 350 that can capture first field of 310 and second field of view 320 as described above. In this embodiment, each of the at least one cameras 350 includes a tilt mechanism that provides a single degree of rotation allowing the at least one camera 350 to tilt to reach a predetermined degree of tilt. As shown in FIG. 3A, the degree of tilt associated with the camera 350 is 0 degrees. As shown in FIG. 3B, the at least one camera 350 is caused to tilt to reach the predetermined degree of tilt which may be 25 degrees. The predetermined tilt angle represents an angles that enables the field of view 310 (210 in FIG. 2) of the camera to cover an entirety of the structural frame of the support structure 140. This allows for the first field of view being captured to tilt to get full coverage of the structure below it. This pivot motion allows for fewer cameras to cover the same space by changing the angle of the images. This can be accomplished through several cameras moving together, or single cameras moving on its own.

As illustrated herein, the tilt function may be accomplished either at point of location of the camera within the housing such that the cameras or camera elements are caused to pivot and tilt while the housing 110 of the imaging device 100 does not move. In another embodiment, the tilt operation can be implemented using a tilt motor that controls the entire position of the housing 110 at the point of connection to the support structure 140 to tilt.

In exemplary operation, the tilt operation can be caused by the camera receiving a tilt control signal issued by an imaging controller embodied in the imaging device 100. This control signal causes the tilt function to be initiated and cause the tilt angle to change from a first tilt angle to the second tilt angle which may be the predetermined tilt angle or any angle between the first tilt angle and the predetermined tilt angle. The control signal can be issued on a schedule or be a single control signal issued in response to user selecting a tilt operation be performed. In addition to the advantages described with respect to static camera arrays, the embodiment shown in FIGS. 3A and 3B provide static cameras which have a degree of tilt allows for reduction in the total number of cameras while still covering the space. Moving an entire array of cameras allows doubling the coverage of a system with the addition of a single motor.

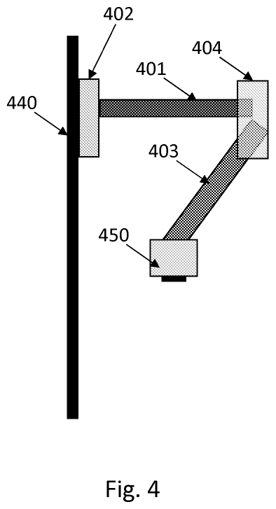

FIG. 4 is another embodiment of the imaging device according to the present disclosure. As shown herein, a support structure 440, which may be similar (or different than support structure 140 described above) is provided. As shown herein, the imaging system includes a first arm 401 having a length L1 and a second arm 403 having a length L2 that are connected to one another via a moveable elbow joint motor 404. An end of the first arm 401 opposite the connection to the second arm is connected to a support structure 440 by a shoulder joint 402 which rigidly attaches the imaging system to the gantry 440. A predetermined length of the first bar 401 is attached to a motor in the shoulder joint 402. An opposite end of the first bar 402 is connected a motor 404 in the elbow joint which couples that end of the first bar to a first end of the second bar 403 extending out from this elbow joint 404. At least one camera and/or camera arrays are connected to an end of the second bar 403 opposite its connection with the elbow joint 404. This arrangement allows coverage of the entire space within a radius of L1+L2 from the shoulder attachment. The system comprising a camera or array of cameras on one or more arms with two degrees of freedom would be installed to cover a single grow area rather than an entire row or room. The single point of rigid attachment allows the system to be wired for communications and power while covering a larger amount of space with a single camera. This implementation may be beneficial if the camera cost is high enough that multiple cameras become prohibitive, or for smaller grow spaces that still need a scanning system.

The imaging system according to the present disclosure is able to cover a large amount of square footage where vegetation is growing using a single device having a plurality of cameras or camera arrays formed integral therewith. An array of cameras allows for minimal complexity to cover the shorter dimension of the grow space while the linear motion system allows the system to cover the entire length of a grow space with a single system. Because the imaging device is battery powered selectively docks with the imager dock, the ease of setup and operation is improved because the imaging device is wireless which allows the system to cover large distances without having to deal with cable management and cost associated therewith. This is particular important in an indoor vegetation growing environment where control of environmental factors is crucial to the success of plant growth and health.

According to the present disclosure, the imaging system and device with its array of stereoscopic cameras advantageously detects an amount of vegetation growth over a large vegetation grow area. The growth measurement may include direct measurement of the height of the plant or the vegetation based on images captured by the array of stereoscopic cameras. In some embodiments, the history of the cultivation as imaged and stored in a memory can be used to determine the growth. In a case where a position the vegetation is in a known arrangement, the position of one or more cameras within the imaging device on, for instance, a rail, can be utilized to determine the growth of the plant or the vegetation. For instance, depth sensing (stereo imaging) can be used with data from more than one camera to determine plant height. This is performed by having multiple cameras that has images overlapped. When images are overlapped, it allows the depth to be triangulated by measuring the difference object location in the images. As long as each camera is calibrated with one another, image analysis on the difference between objects detected in one image from one camera with that of the other image in another camera can be performed. This analysis can be performed with each camera-pair variation to gather more depth calculations to then get a more accurate determination of the real plant height. In another embodiment, different pairs of cameras positioned at different camera positions on the housing of the imaging device can be controlled to capture images representing different distances from the camera. Distance between the cameras impacts the depth resolution, so using cameras which are farther apart is advantageous when the plants are young as compared to older plants that are taller where the use of cameras closer together would be advantageous. Plants closer to the camera require several camera pairs close together in order to detect the height. Cameras that are farther apart have an easier time detecting the difference in variation from one image to the other which gives you a more accurate reading. Younger plants are shorter that we do not need as many cameras (220,320) to cover the entire areas to achieve full coverage. However, as plant grows into older plants, they are much taller. Using the same numbers of cameras (220,320) will not provide full coverage because of the field of view being captured by those cameras. The result is missing some plants because they are too close to the cameras that they won't be captured in the images. By increasing the numbers of the same FOV cameras, it ensures images of all plants are captured. But, by adding a separate set of wide angle FOV cameras (210,310) it reduces the number of cameras needed because the wide angle are able to capture images at a wider field of view.

Embodiment(s) of the present disclosure, in particular the movement of the imaging device and/or cameras and the operation of the cameras, can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like. An I/O interface can be used to provide communication interfaces to input and output devices, which may include a keyboard, a display, a mouse, a touch screen, touchless interface (e.g., a gesture recognition device) a printing device, a light pen, an optical storage device, a scanner, a microphone, a camera, a drive, communication cable and a network (either wired or wireless).

The imaging device (e.g. camera) may include, for example a photomultiplier tube (PMT), a photodiode, an avalanche photodiode detector (APD), a charge-coupled device (CCD), multi-pixel photon counters (MPPC), or other. Also, the function of camera may be realized by computer executable instructions (e.g., one or more programs) recorded on a Storage/RAM.

### Definitions

In referring to the description, specific details are set forth in order to provide a thorough understanding of the examples disclosed. In other instances, well-known methods, procedures, components and circuits have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

The term “about,” as used herein means, for example, within 10%, within 5%, or less. In some embodiments, the term “about” may mean within measurement error.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “includes”, “including,” and “containing” are to be construed as open-ended terms (i.e., meaning “including, but not limited to,”) unless otherwise noted. Specifically, these terms, when used in the present specification, specify the presence of stated features, integers, steps, operations, elements, and/or components, but do not preclude the presence or addition of one or more other features, integers, steps, operations, elements, components, and/or groups thereof not explicitly stated. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

It will be appreciated that the methods and compositions of the instant disclosure can be incorporated in the form of a variety of embodiments, only a few of which are disclosed herein. Variations of those embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. An image capturing system comprising:
a carriage;
at least one camera carried by the carriage; and
at least one motoring wheel to drive the carriage along a rail or a tensioned cable;
wherein the at least one camera is configured to capture images with different angle or field of view.

2. The image capturing system according to claim 1,
wherein the at least one camera captures a plant or a vegetation, and
wherein the at least one camera is configured to capture the images with different angle or field of view based on a growth of the plant or the vegetation.

3. The image capturing system according to claim 1,
wherein the at least one camera captures a plant or a vegetation,
wherein the carriage carries a first camera and a second camera having different field of view, and
wherein whether the first camera performs a capturing of the plant or the vegetation or the second camera performs a capturing of the plant or the vegetation is determined based on a growth of the plant or the vegetation.

4. The image capturing system according to claim 1,
wherein the at least one camera captures a plant or a vegetation,
wherein the at least one camera can be tilted,
wherein a range that the at least one camera is tilted during a capturing of the plant or the vegetation is determined based on a growth of the plant or the vegetation.

5. The image capturing system according to claim 1,
wherein the at least one camera captures a plant or a vegetation,
wherein an angle of the carriage is changeable, and
wherein a range that the angle of the carriage is changed during a capturing of the plant or the vegetation is determined based on a growth of the plant or the vegetation.

6. The image capturing system according to claim 1 further comprising:
a detector to detect a height of a plant or a vegetation.

7. The image capturing system according to claim 5, wherein the detector including depth sensor.

8. The image capturing system according to claim 1, further comprising a collision avoidance sensor.

9. The image capturing system according to claim 1, further comprising a wireless charger configured to charge a battery in the carriage.

10. The image capturing system according to claim 8, wherein the wireless charger is located at an end of the rail.

11. An image capturing device comprising:
a carriage;
at least one camera carried by the carriage; and
at least one motoring wheel to drive the carriage along a fixed path;
wherein the at least one camera is configured to capture images with different angle or field of view.

12. The image capturing device of claim 11, wherein the fixed path is a rail or a tensioned cable.

13. The image capturing device of claim 11, wherein the one or more cameras are movable in a direction orthogonal to direction the carriage travels on the fixed path.

14. The image capturing device of claim 11, wherein the one or more cameras are an array of cameras.

15. The image capturing device of claim 11, wherein the array of cameras can be fixed in position or tiltable.

16. The image capturing device of claim 11, wherein the one or more cameras or array of cameras are attached to a moveable arm.

17. An image capturing device comprising an array of cameras attached to a gantry, wherein the array of cameras can be fixed in position or tiltable.

18. The image capturing device of claim 17, wherein the one or more cameras or array of cameras are attached to a moveable arm.
